(19) United States
(12) Patent Application Publication
(10) Pub. No.: US 2022/0256104 A1
(43) Pub. Date: Aug. 11, 2022

(54) DIGITAL DETECTOR WITH DIGITAL CHARGE INTEGRATION

(71) Applicant: TRIXELL, MOIRANS (FR)

(72) Inventors: Xavier FAVRE, MOIRANS (FR); Martin SIAUD, MOIRANS (FR)

(21) Appl. No.: 17/666,164

(22) Filed: Feb. 7, 2022

(30) Foreign Application Priority Data
Feb. 8, 2021 (FR) 2101174

(51) Int. Cl. H04N 5/32

(52) U.S. Cl. CPC H04N 5/32

(57) ABSTRACT

A digital detector includes a planar sensor and a first monolithic substrate and a second monolithic substrate, the planar sensor being positioned on the first monolithic substrate, the detector comprising, on the first monolithic substrate: a set of pixels organized into a matrix array along rows and down columns and configured so as to generate charges on the basis of radiation; b. column conductors, each connecting the pixels of one same column and intended to carry the charges generated by the pixels; on the second monolithic substrate: c. for each of the column conductors, a charge preamplifier connected to the column conductor, forming a preamplified column conductor that is intended to integrate the charges carried by said column conductor; d. at least one analogue-to-digital converter connected in series to the preamplified column conductors, intended to convert the integrated charges at the output of the charge preamplifiers into a digital voltage; e. a serializer block connected to the at least one analogue-to-digital converter, intended to generate an output voltage.

### CROSS-REFERENCE TO RELATED APPLICATIONS

This application claims priority to foreign French patent application No. FR 2101174, filed on Feb. 8, 2021, the disclosure of which is incorporated by reference in its entirety.

### FIELD OF THE INVENTION

The technical field of the invention is that of medical imaging, and more specifically that of reading out a matrix array of passive pixels typically on tiles of aSi (amorphous silicon) or IGZO (indium gallium zinc oxide). More particularly, it relates to a digital detector and to the integration of the electric charges arising from the pixels via the columns of the matrix array of pixels in order to convert them directly into a digital signal.

In the field of X-ray imaging, there are four distinct constituent elements:
a. the radiation source that emits the X-rays;
b. the filters that modify the properties of the radiation according to the needs of the application;
c. the object to be imaged;
d. the detector that converts the X-ray photons into a digital image.

### BACKGROUND

The invention relates to the digital detector, and more particularly to the conversion of electric charges received at pixel level into an electrical signal that is proportional to the quantity of charges received. The digital detector may implement various possible technologies. One of the technologies employs a scintillator allowing the X-rays to be converted into visible photons and a photodiode matrix array allowing the visible photons to be converted into an electrical signal. It may also be applicable using photoconductors for directly converting X-rays into electrons.

The quality of the image is directly linked to the noise of the electronic chain, a substantial share of which comes from the capacitance of the column of the matrix array of pixels.

Most current systems use straightforward analogue charge-to-voltage conversion circuits associated with analogue-to-digital converters.

First, the standard integrator setups that perform the charge-to-voltage conversion are not optimal with respect to the process of integration and conversion of charges into voltage. They are generally associated with a correction mechanism known as correlated double sampling (CDS) which requires a second, blank integration of the charges present on the column and therefore requires additional operating time which is detrimental in terms of execution speed.

Second, the analogue-to-digital converter located downstream requires an analogue image chain allowing the analogue output signal to be adapted into an input voltage for the analogue-to-digital converter. Faults due to the association of the two blocks result. Previously external, which resulted in a build-up of parasitic effects at each interface, the trend is now to integrate the analogue-to-digital converter within the same circuit, but the two functions remain dissociated with analogue-to-digital converter blocks that require a multiplexing function.

The solutions proposed in the prior art are essentially based on the use of CDS (correlated double sampling).

The solutions that do not comprise any preamplifier require the use of a correction with a detrimental impact on the integration time since the column has to be read out twice to eliminate undesirable effects. Specifically, a correction of CDS type requires that the column be reset in order to perform a blank readout thereof, which is then subtracted from the signal. This reset generates new kTC noise which has to be filtered. Low-pass filtering is used to reduce this column noise; however, the time constants required for effective filtering are not compatible with the pixel convergence times, hence the impact on the readout time for the matrix array of pixels.

In the case of a readout with a charge preamplifier associated with a column, the component can integrate the charges from the column in the minimal time needed to transfer the charges from the pixel to the column.

In the case of a readout in CDS mode with an integrator without a charge preamplifier, additional times are needed to reset the column and to sample the signal before the integration of the charges from the column in the minimal time needed to transfer the charges from the pixel to the column.

In other words, the solutions of the prior art propose converting charges into analogue voltage in the column, followed by multiplexing of the various columns to an analogue-to-digital conversion block. The analogue data are multiplexed to the analogue-to-digital converter. Having an independent ADC circuit generates additional distortions and noise. Documents U.S. Pat. Nos. 5,184,018 A and 6,642,494 B1 disclose a matrix-array detector of the prior art. In both cases, the system uses a circuit without a charge preamplifier upstream, between the sensor composed of the matrix array of pixels and the processing chain, with a block ADC and analogue multiplexer, the ADC being located off the substrate of the processing circuit. The document by Simoni and al “A digital vision sensor” (XP027220184) discloses a sensor comprising one analogue-to-digital converter per channel, not comprising any preamplifier, and aiming to convert the current from an embedded photodiode generating significantly large currents.

A block or monolithic ADC requires multiplexing of the channels at input and its conversion time is equal to a row time divided by as many channels as the matrix array of pixels (this can typically range from 1000 to 5000 channels, possibly more), so that the total conversion time is transparent with respect to the readout time for the matrix array of pixels and chaining together the rows and the images without latency. A parallel ADC integrated into the column allows the conversion time to be reduced to just one row time since there is one ADC per processing channel. This results in an entirely particular ADC design because besides the initial resolution which remains the same, the conversion speed is completely different in each of the two cases and the consumption and area constraints vary hugely.

It should be noted that producing such an ADC is not straightforward because it is on the border between two types de converters: slow with very high resolution and fast with low resolution. The characteristics required of the ADC for such an application correspond to a trade-off between a medium resolution associated with a high speed.

### SUMMARY OF THE INVENTION

The invention aims to overcome all or some of the problems mentioned above by providing a digital detector that combines a charge preamplifier and an analogue-to-digital converter within a single processing channel, all inside one same monolithic integrated circuit. Such an association makes it possible to reduce the kTC noise caused by the column and to minimize the noise of the chain downstream. The preamplifier makes it possible to reduce the specific column noise known as kTC. The integration of the ADC and the combination thereof with the charge-to-voltage converter makes it possible in turn to reduce parasitic noise by reducing the processing chain and by avoiding the multiplexing of the data at input. The result is transmitted in the form of a serialized digital voltage, thereby ensuring that the signal is not subject to subsequent deterioration. The invention allows the rapid integration of charges from the matrix array of pixels while generating a minimal amount of signal interference.

The challenge in implementing the invention with a parallel ADC is that of integrating an ADC, which is a substantial block, and of combining it with the unitary routing of the channel (column): in addition to the particular production complexity, the area and consumption become critical since there are as many ADCs as there are columns in the matrix array of pixels.

To that end, the subject of the invention is a digital detector comprising a planar sensor, a first monolithic substrate and a second monolithic substrate, the planar sensor being produced on the first monolithic substrate, the detector comprising:

on the first monolithic substrate:
a set of pixels organized into a matrix array along rows and down columns and configured so as to generate charges on the basis of radiation impinging on the detector;
column conductors, each connecting the pixels of one same column and intended to carry the charges generated by the pixels;
   on the second monolithic substrate:
for each of the column conductors, a charge preamplifier connected to the column conductor, forming a preamplified column conductor that is intended to integrate the charges carried by said column conductor;
at least one analogue-to-digital converter connected in series to the preamplified column conductors, intended to convert the integrated charges at the output of the charge preamplifiers into a digital voltage;
a serializer block connected to the at least one analogue-to-digital converter, intended to generate an output voltage on the basis of the digital voltages from the at least one analogue-to-digital converter.

Advantageously, one from among the at least one analogue-to-digital converter is connected to each preamplified column conductor.

Advantageously, at least two of the preamplified column conductors are connected together, and the at least two preamplified column conductors connected together converge towards one from among the at least one analogue-to-digital converter.

Advantageously, the serializer block is positioned on the second monolithic substrate, downstream of the at least one analogue-to-digital converter.

### BRIEF DESCRIPTION OF THE DRAWINGS

The invention will be better understood and further advantages will become apparent from reading the detailed description of one embodiment provided by way of example, which description is illustrated by the attached drawing, in which:

FIG. 1 schematically shows a conventional image detector;

FIG. 2 schematically shows an image detector of the prior art and the detail of a charge integration architecture of an image detector of the prior art;

FIG. 3 schematically shows an image detector according to the invention and the detail of a charge integration architecture of an image detector according to the invention.

For the sake of clarity, these figures are not all to the same scale. Moreover, the same elements will bear the same references in the various figures.

### DETAILED DESCRIPTION

Generally speaking, the invention refers to a conventional image detector typically comprising a planar sensor comprising a set of pixels organized into a matrix array along rows and down columns, row addressing blocks, column readout blocks, row conductors connecting the rows of pixels to a row addressing block, and column conductors connecting the columns of pixels to a column readout block. It should be noted that, in the context of the present patent application, the concepts of column and row have only a relative meaning, with a row of pixels and a column of pixels being nothing more than rows of pixels that are arranged for example, and without limitation, perpendicularly to one another. A row conductor, respectively column conductor, is defined as being oriented parallel to a row of pixels, respectively a column of pixels.

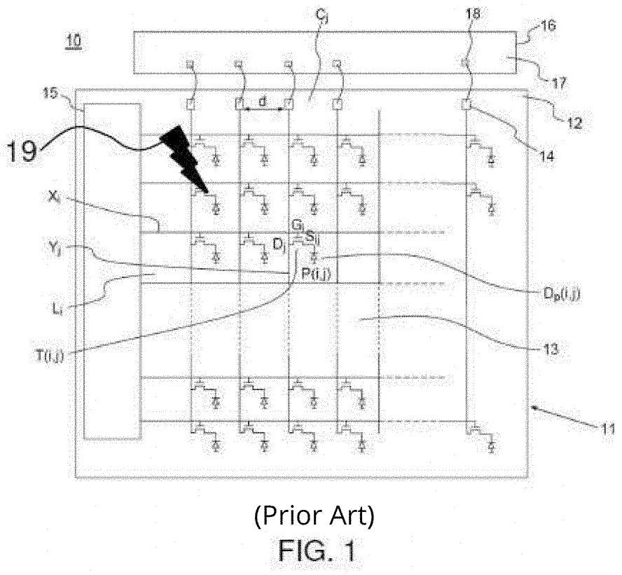

FIG. 1 shows a conventional image detector 10. The image detector 10 comprises a sensor 11 formed on a first monolithic substrate 12. The first monolithic substrate 12 comprises a set of pixels P(i,j) organized into a matrix array 13 along rows Li and down columns Cj. The matrix array 13 may contain any number of rows and columns thus forming pixels P(i,j). The matrix array 13 forms a geographical region on the first substrate 12. The pixels are denoted in the generic form P(i,j), where i and j are natural integers denoting the rank of the row and the rank of the column in the matrix array 13, respectively. The set of pixels P(i,j) is configured so as to generate signals on the basis of radiation impinging on the detector 10. The sensor 11 comprises column conductors Yj, each connecting the pixels of one same column Cj. The column conductors Yj are intended to carry the signals generated by the pixels P(i,j). Likewise, the sensor 11 comprises row conductors Xi, each connecting the pixels of one same row Li. The matrix array 13 of pixels P(i,j) comprises columns Cj of even ranks and of odd ranks. Likewise, the matrix array 13 of pixels P(i,j) comprises rows Li of even ranks and of odd ranks. The sensor 10 comprises contact pads 14 located at the edge of the first substrate 12 and outside of the matrix array 13 of pixels P(i,j). The contact pads 14 are connected to the column conductors Yj. The image detector 10 comprises a row addressing block 15 located close to the first substrate 12 and connected to the row conductors Xi. Row addressing block 15 is the name given to any assembly comprising at least one row addressing block. The block 15 may be integrated into the first substrate 12, as shown in FIG. 1, or else integrated into a different substrate. The row addressing block 15 makes it possible to address each row of pixels Li individually. The image detector 10 comprises a column readout block 16 that is generally formed on a second substrate 17 different from the first substrate 12. The column readout block 16 comprises connection points 18 that connect the column readout block 16 to the contact pads 14. The column readout block 16 makes it possible to read out the signals generated by the pixels of the row selected by the row addressing block.

A pixel P(i,j) comprises a photodiode Dp(i,j) associated with an electronic switch T(i,j). The photodiodes Dp(i,j) may of course be replaced with any photosensitive element able to generate an electrical signal when it is subjected to photon radiation. The pixel structure shown in FIG. 1 is intentionally simplified, and more complex structures may be implemented within the scope of the invention.

The switch T(i,j) formed by a transistor is connected by its gate Gi to the row conductor Xi of the row i, by its drain Dj to the column conductor Yj and by its source Sij to the cathode of the photodiode Dp(i,j). The anodes of all of the photodiodes Dp(i,j) are connected to a common potential, for example ground. The row addressing block 15 comprises elements for generating the signals to be injected onto the row conductors Xi in order to drive the opening and closing of the transistors T(i,j). The column readout block 16 may comprise elements for processing the signals received on the column conductors Yj. These may in particular be an amplifier and/or an analogue-to-digital converter.

The image detector 11 conventionally operates as follows. In an image capture phase, the exposure of the photodiodes Dp(i,j) to radiation generates electric charges at the source Sij. The quantity of charges at each source Sij depends on the intensity of the radiation received by the pixel P(i,j) under consideration. The image capture phase is followed by a readout phase, performed row by row. The signals injected onto the various row conductors Xi move successively to the active state, such that the potential of each column conductor Yj is successively representative of the quantity of electric charges that have built up in the various pixels P(i,j) of the column j.

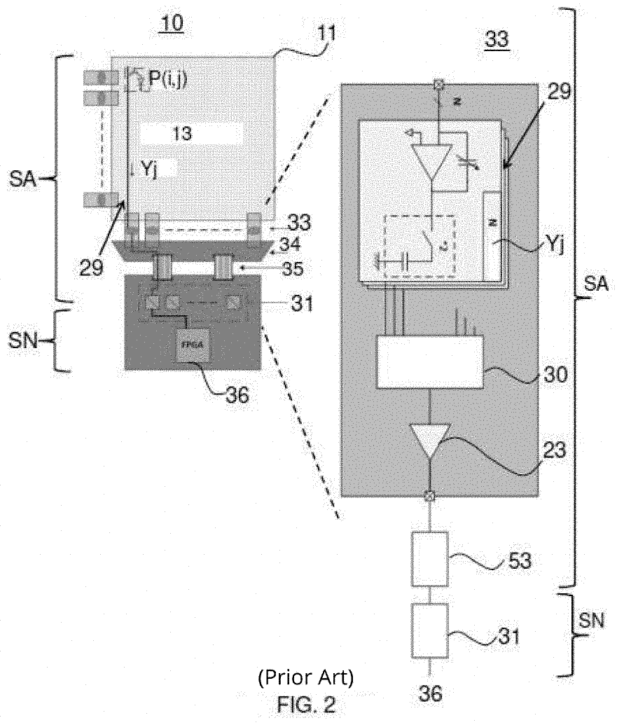

FIG. 2 schematically shows an image detector 10 of the prior art and the detail of a charge integration architecture of an image detector of the prior art. The digital detector 10 of the prior art comprises a planar sensor 11 formed on a first monolithic substrate. The planar sensor comprises a set of pixels P(i,j) organized into a matrix array 13 along rows Li and down columns Cj and configured so as to generate charges on the basis of radiation 19 impinging on the detector 10. The sensor 11 comprises column conductors Yj, each connecting the pixels P(i,j) of one same column Cj and intended to carry the charges generated by the pixels P(i,j). At this stage, the signal is analogue. At the output of the sensor 11 is located a set of integrated readout circuits 33 with analogue output. These circuits 33 are connected to a daughterboard 34, which is connected by board connectors 35 to analogue-to-digital converters 31. In other words, the signal is processed in an analogue manner outside of the first substrate. The analogue signal region is represented by the reference SA. The digital processing, represented by the reference SN, takes place after the output of the analogue-to-digital converters 31.

The integration of the charges at the foot of the column is described with the aid of the detail shown on the right-hand side of the figure.

In this charge integration architecture of the detector 10 of the prior art, each of the N columns is connected to an analogue multiplexer 30, which is itself connected to an analogue-to-digital converter block 31. In other words, the charges from the N channels, i.e, N column conductors Yj, are integrated and sampled (29), and then multiplexed in an analogue manner to one or more external analogue-to-digital converter blocks 31. The conversion of the data is performed outside of the architecture and requires signal matching (gain, buffering 53, etc.). For each channel, a serializer block 23 allows the signals to be serialized to the output, for example to an FPGA circuit 36. Lastly, a CDS correction is applied, i.e., in the architecture of the prior art, there is a second readout with a prior reset of the column in order to perform a blank readout thereof which is then subtracted from the signal.

In this architecture of the prior art, the conversion of the charges into analogue voltage is performed in the columns. Next, outside of the columns, multiplexing of the various columns to the analogue-to-digital converter block is carried out. The analogue data are multiplexed to the converter.

In addition to requiring a multiplexing function, this solution of the prior art requires additional operating time which may be detrimental in terms of execution speed due to the correlated double sampling. Additionally, the analogue processing of the signal downstream of the column does not allow the integrity of the data to be ensured.

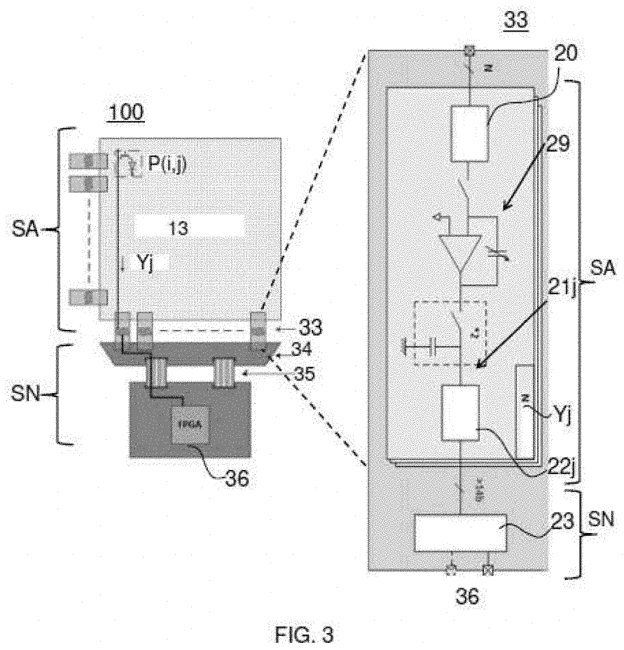

FIG. 3 schematically shows an image detector 100 according to the invention and the detail of a charge integration architecture of the image detector according to the invention. The basic description of the digital detector 100 is similar to that of the detector 10 of FIG. 1. The differences appear at the level of the integration block, shown here in greater detail on the right-hand side of the figure. The references hereinafter, even if they are not all present in FIG. 3, are used on the basis of those elements in common with the detector of FIG. 1 and of FIG. 2 for ease of understanding. The digital detector 100 according to the invention comprises a planar sensor 11 and a first monolithic substrate and a second monolithic substrate, the planar sensor 11 being positioned on the first monolithic substrate. The planar sensor comprises a set of pixels P(i,j) organized into a matrix array 13 along rows Li and down columns Cj and configured so as to generate charges on the basis of radiation 19 impinging on the detector 100. The sensor 11 comprises column conductors Yj, each connecting the pixels P(i,j) of one same column Cj and intended to carry the charges generated by the pixels P(i,j). According to the invention, the detector comprises, on the second monolithic substrate, which is distinct from the first substrate, for each of the column conductors Yj, a charge preamplifier 20 connected to the column conductor Yj, forming a preamplified column conductor 21j that is intended to integrate the charges carried by said column conductor Yj. The sensor 11 comprises at least one analogue-to-digital converter 22j connected in series to the preamplified column conductors 21j, intended to convert the integrated charges at the output of the charge preamplifiers 20 into a digital voltage. Lastly, the sensor 11 comprises a serializer block 23 connected to at least one analogue-to-digital converter 22j, intended to generate an output voltage on the basis of the digital voltages from the at least one analogue-to-digital converter 22j. As can be seen in FIG. 3, the signal is processed in an analogue manner on the second monolithic substrate. The analogue signal region is represented by the reference SA. The digital processing, represented by the reference SN, takes place at the output of the first monolithic substrate.

In the digital detector of the invention, the serializer block 23 is positioned on the second monolithic substrate, downstream of the at least one analogue-to-digital converter 22j.

The analogue-to-digital conversion is carried out on each channel in parallel before the serialization on one or more digital outputs, this serialization being able to be likened to digital multiplexing. One particularity of the invention lies in the combination of the charge preamplifier and of the analogue-to-digital converter per channel upstream of the serializer block on a single monolithic substrate dissociated from the photodiode matrix array.

The principle of the invention is thus based on a monolithic circuit that carries out the conversion of charges into digital voltage. In other words, the digitization takes place in each column, before multiplexing. The invention thus makes it possible to perform digital multiplexing that is less sensitive to the noise and to the interference in the analogue signals. This digital multiplexing is made possible through the use of sigma-delta converters in each column. In other words, the invention aims to convert X-rays into charges via a special sensor and then to amplify this charge (of the order of a pC) in order to digitize it, by means of a preamplification and integration stage.

The invention allows conversion at more than 16 bits, on matrix arrays that are typically of very large sizes (more than a million pixels) for an equivalent number of frames.

Unlike the prior art which proposes a solution in which analogue multiplexing is followed by digitization, the invention proposes positioning the analogue-to-digital converter on each channel and then performing digital multiplexing. The invention consists in merging, or combining, the ADC on the second substrate. This results in routing of the channel with the rest of the blocks.

In a first embodiment of the invention, the solution consists in associating, for each channel, a charge preamplifier 20 and an analogue-to-digital converter (ADC) 22j in one same monolithic integrated circuit. In other words, an analogue-to-digital converter 22j is connected to each preamplified column conductor 21j.

The integration of the analogue-to-digital converter onto the first substrate and directly with the preamplified column conductor allows the number of interfaces between the analogue domain and the digital domain to be reduced. This solution allows the direct generation of one or more digital outputs from the first chip, without risk of degradation of the signal after output thereof. Lastly, this direct integration allows simplification of the circuit boards in the subsequent processing of the signal.

Thus, this integration allows a significant improvement in linearity, noise and crosstalk performance while reducing the cost and the bulk of the components that perform the integration/digitization function.

In another embodiment of the invention, each channel is associated with a charge preamplifier and one analogue-to-digital converter (ADC) 22j is associated with a plurality of channels in one same monolithic integrated circuit. In other words, an analogue-to-digital converter 22j is connected to a plurality of preamplified column conductors 21j. This variant shares the analogue-to-digital converter between a plurality of channels for the sake of saving space and reducing density when the application allows it.

In one variant of the invention, at least two of the preamplified column conductors 21j (for example four column conductors) may be connected together, and the at least two preamplified column conductors 21j connected together converge towards one from among the at least one analogue-to-digital converter 22j.

The invention is based on producing a converter for converting electric charges from a matrix array of pixels directly into a digital voltage on the second monolithic substrate. As can be seen in FIG. 3, at the output of the sensor, the signal is digital, and the integrity of the data of this signal is thereby ensured.

The invention differs from the prior art in the association, for each channel or at least for a plurality of channels, of a charge preamplifier and an analogue-to-digital converter (ADC) in one same monolithic integrated circuit. This integration allows a significant improvement in linearity, noise and crosstalk performance while reducing the cost and the bulk of the components that perform the integration/digitization function.

More precisely, the charge preamplifier overcomes the limitations of standard integration stages by allowing the charge to be integrated at the same rate as the transfer of the charge from the pixel via the column to the input of the integrator circuit. This arrangement has two advantages. The first advantage is the absence of time loss: there is a significant time saving in overall processing time and therefore in readout speed for the tile and transfer time (the data processing time is shorter than the integration time). The second advantage is a significant reduction in noise as a result of the integration of the input charge, in particular at high gain.

In addition, the direct association of the preamplifier directly with the analogue-to-digital converter ADC makes it possible to omit the conventional analogue multiplexing stage between a group of columns and its single converter.

The integration of the analogue-to-digital converter ADC inside the processing channel makes it possible to have an analogue-to-digital converter ADC that is perfectly designed and adapted for the situation and thereby limit all parasitic effects. The advantages of this integration are manifold:
a. very significant improvement in overall integral linearity;
b. very significant improvement in spatial linearity (in particular channel-to-channel);
c. significant reduction in noise performance;
d. limiting channel-to-channel vertical crosstalk effects (each processing channel is independent until final digitization);
e. overall solution cost reduction by virtue of the integration.

However, drawbacks should be taken into account. In particular, there is the difficulty in developing and in integrating an analogue-to-digital converter ADC that is compatible with applications that require high resolution (14-16 bits) and such performance levels (speed, linearity, noise). In addition, the solution proposed by the invention requires that the area of the chip be increased, and therefore with it the cost of the chip. Lastly, the solution proposed by the invention entails an increase in the consumption of the chip, requiring suitable heat dissipation.

the invention makes it possible to decrease the kTC noise caused by the column by virtue of the particular setup of the charge preamplifier combined with an analogue-to-digital converter (ADC) in order to minimize the noise of the chain downstream. The result is transmitted in the form of a serialized digital voltage (and there is no more deterioration of the signal after this step).

The invention makes it possible to address the need of being able to integrate as quickly as possible the charge from the matrix array of pixels while generating the least possible interference in the signal (noise/linearity/leakage/crosstalk), which the charge preamplifier makes possible by associating it with an ADC within one same block on the same chip. The ADC and the transfer speed of the digital output should also be fast enough with respect to the characteristics of the matrix array of pixels (pixel convergence time in particular).

In the detector 100 of the invention, the analogue-to-digital converter is placed inside the column. There is no longer any multiplexer in this architecture, which allows time to be saved. The analogue processing is performed in the column and delivers a digital voltage at the column output. Here, the concept of column should be understood as being extended beyond the physical column on the photodiode matrix array and comprising the individual “channels” in the conversion circuit external to the matrix array. This eliminates the problems of signal contamination or stability. At the column output, the detector of the invention allows digital signal processing, which ensures the integrity of the image of the analogue datum from the sensor.

The particularity of the invention is based on the direct conversion of charges into a digital signal, without alteration of the signal by intermediate steps and/or stages. The invention is based on coupling the charge preamplifier to an analogue-to-digital converter ADC in the same processing channel of a column of pixels.

Designing an analogue-to-digital converter and using it in parallel makes it possible for the charge to be directly converted into a digital signal without any other intermediate step (analogue multiplexing in particular) and therefore to limit sources of noise and distortion.

Associating the preamplifier with such a parallel analogue-to-digital converter ADC thus makes it possible to create a true “charge-to-digital” converter (charge digitizer), the entirety of the conversion taking place in the processing channel of the column and requiring no additional operations in analogue form such as, for example, a multiplexing operation.

The analogue-to-digital converter ADC has to be parallel (i.e. there is one analogue-to-digital converter ADC per channel/preamplifier or alternatively per group of channels/preamplifiers) and integrated into the immediate processing chain of the column. The analogue-to-digital converter ADC may be of any type (in particular of successive-approximation or sigma-delta type), and the resolution does not matter (in general 14 or 16 bits, possibly more).

Depending on the requirements of the target applications (speed, noise/linearity, size of the pixels and binning possibilities), and as already mentioned, it is possible to consider one parallel analogue-to-digital converter in common for a plurality of channels in order to optimize the design of the circuit in terms of area.

The principle of the invention may be applied to all types of imagers with passive pixels (1 T imager), in particular medical or NDT applications.

1. A digital detector comprising a planar sensor and a first monolithic substrate and a second monolithic substrate, the planar sensor being positioned on the first monolithic substrate, the detector comprising,
on the first monolithic substrate:
a. a set of pixels (P(i,j)) organized into a matrix array along rows (Li) and down columns (Cj) and configured so as to generate charges on the basis of radiation impinging on the detector;
b. column conductors (Yj), each connecting the pixels (P(i,j)) of one same column (Cj) and intended to carry the charges generated by the pixels (P(i,j));
on the second monolithic substrate, which is distinct from the first substrate:
c. for each of the column conductors (Yj), a charge preamplifier connected to the column conductor (Yj), forming a preamplified column conductor that is intended to integrate the charges carried by said column conductor (Yj);
d. at least one analogue-to-digital converter connected in series to the preamplified column conductors, intended to convert the integrated charges at the output of the charge preamplifiers into a digital voltage;
e. a serializer block connected to the at least one analogue-to-digital converter, the at least one analogue-to-digital converter being arranged upstream of the serializer block on the second monolithic substrate, the serializer block being intended to generate an output voltage on the basis of the digital voltages from the at least one analogue-to-digital converter.

2. The digital detector according to claim 1, wherein one from among the at least one analogue-to-digital converter is connected to each preamplified column conductor.

3. The digital detector according to claim 1, wherein at least two of the preamplified column conductors are connected together, and the at least two preamplified column conductors connected together converge towards one from among the at least one analogue-to-digital converter.

4. The digital detector according to claim 1, wherein the serializer block is positioned on the second monolithic substrate, downstream of the at least one analogue-to-digital converter.
